(19) United States
(12) Patent Application Publication
(10) Pub. No.: US 2017/0056381 A1
(43) Pub. Date: Mar. 2, 2017

(54) PYRIDOXAMINE FOR THE TREATMENT OF SICKLE CELL DISEASE, THALASSEMIA AND RELATED BLOOD DISEASES

(71) Applicants: NANOMETICS LLC, New York, NY (US); ALBERT EINSTEIN COLLEGE OF MEDICINE, INC., Bronx, NY (US)

(72) Inventors: Steven Isaacman, New York, NY (US); Seetharama A. Acharya, Cresskill, NJ (US)

(21) Appl. No.: 15/120,206

(22) PCT Filed: Feb. 20, 2015
(86) PCT No.: PCT/US2015/016753

§ 371 Date: Aug. 19, 2016

Related U.S. Application Data
(60) Provisional application No. 61/942,397, filed on Feb. 20, 2014.
(60) Provisional application No. 62/008,018, filed on Jun. 5, 2014.

(51) Int. Cl. A61K 31/44; A61K 45/06; A61K 9/00

(52) U.S. Cl. CPC A61K 31/44; A61K 9/0053; A61K 45/06

(57) ABSTRACT

The present invention relates to treatments and therapies for anemia conditions and diseases of the blood, and more particular is a therapy for the acute and chronic treatment of sickle cell diseases and thalassemia by administration of pyridoxamine, or a pharmaceutically acceptable salt thereof, optionally in combination with an additional bioactive agent.

### RELATED APPLICATIONS

This application claims the benefit of priority of U.S. provisional application No. 61/942,397, filed Feb. 20, 2014, entitled “Pyridoxamine for the Treatment of Sickle Cell Anemia, and U.S. 62/008,018, filed Jun. 5, 2014 of identical title, the entire contents of both applications being incorporated by reference in their entirety herein.

### FIELD OF THE INVENTION

The present invention relates to treatments and therapies for anemia conditions and diseases of the blood, and more particular is a therapy for the acute and chronic treatment of sickle cell diseases and thalassemia by administration of pyridoxamine, or a pharmaceutically acceptable salt thereof, optionally in combination with an additional bioactive agent.

### BACKGROUND OF THE INVENTION

Sickle cell disease (SCD) is a global health issue that affects over 13 million people worldwide, including ˜100,000 Americans. [1, 2] SCD results from an autosomal recessive red blood cell (RBC) disorder and is most common in populations of African, Mediterranean or Asian ancestry. [1, 2] Over 300,000 babies are born each year with the disease. [1] SCD is caused by an inherited hemoglobinopathy that weakens the oxygen binding affinity of hemoglobin (Hb), enabling polymers of deoxyhemoglobin (deoxyHb) to form in the erythrocyte. Consequently, the RBCs become rigid and assume distorted morphologies. [2] The distorted erythrocytes are prone to hemolysis, which disrupt nitric oxide (NO) homeostasis and causes ischemia-reperfusion injury, and rapid clearance from the circulation, which results in anemia. The compromised erythrocytes can occlude the circulation and contribute to a cascade of events that culminate in vaso-occlusion (VOC) and painful episodes of sickle crisis. VOC can result in ischemia, infarction, hemolytic anemia, organ damage and other debilitating acute and chronic effects. [2] While the frequency, severity and duration of VOC can vary among individuals, the recurrent episodes are characteristically painful, and lead to a high rate of hospitalization. [3] Consequently, SCD exhibits significant financial stress on the United States Healthcare system, with annual costs exceeding $1.1 billion. [3]

Current paradigm for treatment: SCD can be cured using hematopoietic stem cell transplants, and the severity of the disease can be reduced with repeated transfusions. However, these approaches are costly and associated with high levels of risk. The only FDA approved therapeutic for SCD is hydroxyurea (HU). HU induces fetal Hb (HbF) production, and has a strong clinical track record in children and adults. Proper use of HU requires strict compliance; self-administration by the patient; and careful monitoring by the physician to ensure proper dosing schedules. [2] Unfortunately, approximately a third of adult patients do not respond to HU treatment. [4] Moreover, myeolsuppression, reproductive toxicity and possible carcinogenic affects are all serious side effects associated with long-term use. [5] Importantly, there is currently no commercial therapeutic to avert imminent episodes of crisis or to attenuate acute events of crisis once they have begun. Patients are typically prescribed medications that alleviate the pain associated with episodes of crisis.

### SUMMARY OF THE INVENTION

The present invention overcomes deficiencies of the presently available treatments and therapies by providing a novel therapy for sickle cell diseases, Thalassemia and other related blood diseases (e.g. sickle cell trait) comprising oral administration of a therapeutically effective amount of pyridoxamine, or pharmaceutically acceptable salts of pyridoxamine, to patients in need. The pyridoxamine compound can be delivered by any means, preferably orally or intravenously, and is useful as a chronic treatment and as an acute treatment. Accordingly, the present invention is directed to a method of treating and/or reducing the likelihood of a blood disease, including sickle cell disease, Thalassemia or a related blood discussed comprising administering to a patient in need an effective amount pyridoxamine or a pharmaceutically acceptable salt.

Chronic hemolytic anemia and vaso-occlusive ischemia-reperfusion like injury are the hallmark pathologies of sickle cell disease. [5] Oxidative stress and nitric oxide (NO) homeostasis are thought to play a major role in the severity of the disease, and agents that modulate these pathways are highly desirable. The multifactorial process, which leads to VOC involves oxidative stress, damages to red blood cells (RBC), inflammation, vascular leukocyte adhesion, coagulation and abnormal rheology, and vascular tone modulation. [6] Ameliorating oxidative stress by directly targeting free radicals, reactive carbonyls and other oxidizing species with non-toxic therapeutic agents has tremendous potential as a treatment for SCD. Pyridoxamine can act by several potential anti-oxidative mechanisms: nucleophilic scavenging of reactive carbonyl species; trapping of free radicals; and chelation of radical generating metal ions. [18, 19] In Berkeley models of SCD, a single dose of pyridoxamine caused normalization of both deoxyHb/oxyHb ratio and cerebral blood flow (CBF), with maximum effect observed at 72 hours. This indicates potential for long-term benefits associated with an appropriate chronic dosing schedule. Pyridoxamine for treatment of SCD represents a significant advancement because the molecule is multimodal, acting by several potential anti-oxidative mechanisms [18, 19] and simultaneously ameliorating multiple pathologies. This is thought to culminate in a prolonged benefit to the overall health of the microvasculature system that is detectable for several days, even after the drug is no longer present in the system. Studies to fully elucidate the mechanism associated with this prolonged therapeutic effect are currently underway. Pyridoxamine also has potential as an acute therapy for sickle crisis, for which, there are no FDA approved therapeutic options. In both Berkeley and NY1 DD mouse models exposed to hypoxic stress, pyridoxamine preserved endothelial function and ameliorated the hypoxia-induced endothelial activation that precedes venular occlusion and episodes of sickle crisis. In these studies, significant improvements in leukocyte behavior and hemodynamic properties were observed. These preclinical results are encouraging, and support the hypothesis that pyridoxamine will be useful as a therapy to promote normal microvascular function. When used prophylactically, it is anticipated that chronic dosing with pyridoxamine will attenuate the damage to red blood cells and the endothelium, thus normalizing the hemodynamic properties of the patient. As an acute intervention, pyridoxamine is expected to be useful to avert imminent episodes of sickle crisis. This hypothesis is supported by analysis with intravital microscopy in both Berkeley and NY1 DD models, where pyridoxamine ameliorated the effects of hypoxia/reoxygenation to near wild type levels.

In one embodiment, the present invention provides pharmaceutical compositions comprising (a) a dosage unit of 10 mg to 3000 mg of pyridoxamine, or a pharmaceutically acceptable salt thereof; and (b) a pharmaceutically acceptable carrier, optionally in combination with an additional bioactive agent, preferably an active agent for treating sickle cell disease, Thalassemia and other related blood diseases as otherwise described herein. These additional bioactive agents can include anti-sickling agents, selectin inhibitors, fetal hemoglobin regulators (including fetal hemoglobin inducing agents), agents which inhibit a platelet ADP receptor, agents which increase the affinity of sickle hemoglobin's binding to oxygen, anti-oxidant agents, anti-inflammatory agents, agents that target leukocyte adhesion and anti-platelet agents, among others as described herein.

In certain embodiments, the present invention provides pharmaceutical compositions comprising: (a) pyridoxamine, or a pharmaceutically acceptable salt thereof; and (b) one or more additional bioactive agents as described herein, preferably a compound selected from the group consisting of fetal hemoglobin-inducing agents, agents that target leukocyte adhesion, anti-inflammatory agents, anti-oxidant agents, anti-platelet agents and anti-sickling agents, among others.

In a preferred embodiment, the one or more compounds are selected from the group consisting of hemoglobin-inducing agents, in particular fetal hemoglobin-inducing agents, but not limited to, hydroxyurea, sodium phenyl butyrate, sodium phenyl acetate, sodium phenyl propionate, or an alternative pharmaceutical salt thereof (as disclosed in U.S. Pat. No. 5,712,307, which is incorporated by reference herein), cyclic peptides such as FK228 (depsipeptide) and analogs thereof, as disclosed in Anemia 2012; 2012: 428137 (published online May 14, 2012), benzamides (such as MS-275); non cyclic and cyclic hydroxamates, for example SAHA (suberoylanilide hydroxamic acid) and TSA (Trichostatin A) among others.

In a further embodiment, the present invention provides methods for treating sickle cell disease and/or complications arising from sickle cell disease including, comprising administering to the human patient an amount of pyridoxamine, or a pharmaceutically acceptable salt thereof, effective to alleviate the physiological manifestations of the disease including, but not limited to, venular occlusion and sickle crisis and chronic and systemic vasculopathies including stroke, sickle retinopathy, open leg ulcers, multi-organ failure, aseptic bone necrosis, dactylitis, hepatopathy, splenic autoinfarction, pulmonary hypertension, cognitive deficits, renal failure, cholecystitis, decreased fertility, increased susceptibility to infection and decreased opsonization, among others.

In yet another embodiment, the present invention provides methods for treating or averting episodes of sickle cell crisis whereby pyridoxamine, or a pharmaceutically acceptable salt, is administered orally or by injection to mitigate the crisis episode.

In addition to the acute events that are normally associated with sickle cell disease, secondary disease states and conditions such as vessel occlusion can also cause chronic and systemic vasculopathies that yield devastating results. These can include: stroke, sickle retinopathy, open leg ulcers, multi-organ failure, aseptic bone necrosis, dactylitis, hepatopathy, splenic autoinfarction, pulmonary hypertension, cognitive deficits, renal failure, cholecystitis and decreased fertility. Increased susceptibility to infection and decreased opsonization capabilities have caused significant mortality in this population. To date, therapy has focused on blood transfusions and hydroxyurea. However, transfusion begets chronic iron overload and the necessity of chelation therapy (to avoid cirrhosis and cardiac failure due to hemosiderosis) and the exposure of the patient to a high likelihood of alloimmunization. Hydroxyurea has been of major benefit to patients, both in reducing overall mortality and in decreasing painful crises. In addition to increasing fetal hemoglobin (HbF) levels, hydroxyurea can also increase nitric oxide (NO) levels and has been shown to depress leukocyte counts; decreasing the local inflammatory reaction and increasing mean cell volume. The complexity of SCD dictates that a multimodal approach to treat patients will likely be needed.

The present invention which relies on the use of pyridoxamine or a pyridoxamine salt provides for a non-toxic, therapeutic, preferably including an oral therapeutic, that will attenuate ongoing oxidative stress in the microcirculation, improve perfusion to organs, and mitigate the damage to circulating RBC. It is anticipated that the present invention will be useful in both children and adults, as a single agent or as part of a combination therapy.

As a prophylactic, it is expected that pyridoxamine will reduce oxidative stress mediated complications in the microcirculation, hence improving vascular and organ health in SCD patients. While the prophylactic approach would have a positive impact on all SCD patients, it could have maximum impact in children where progressive damage to endothelium and organs could be significantly mitigated. [13, 14] From a purely cost perspective, episodes of crisis often require emergency room visits and extended hospital stays that on average exceed 5 days and cost between $5K and $10K per visit. [3] Medicaid and Medicare are the major payers, and responsible for covering up to 75% of the related hospital costs, resulting in annual total costs to the United States healthcare system in excess of $1 billion. [3] The low toxicity and multimodality of pyridoxamine could render the therapeutic useful as a complement or supplement to HU and other therapies. Pyridoxamine is expected to have a broad impact on global public health, and be useful as an inexpensive oral medication to attenuate oxidative damage to RBC, normalize the microcirculatory system and ameliorate the likelihood of VOC.

The pathophysiology of SCD resembles many aspects of ischemia-repurfusion injury. Oxidative stress is a major driver of the pro-inflammatory state associated with SCD. [7] Higher rates of autoxidation of HbS in vivo, RBC damage and lysis, cell free hemoglobin, ischemia-reperfusion injury and inflammation all contribute to increased levels of oxidants, which perpetuate the pro-inflammatory state. Cell free hemoglobin, as a result of chronic hemolysis, reduces nitric oxide (NO) bioavailability, which has vasoconstrictive, antithrombotic and anti-inflammatory properties. [20] It also causes non-enzymatic activation and damage of the endothelium by incorporation of free heme into endothelial cell membranes and generating reactive oxygen species (ROS). [21] Endothelial generation of ROS is also thought to damage erythrocyte cell walls, further promoting hemolysis. [7] Chronic and acute instances of VOC result in ischemia-reperfusion injury, which is characterized by generation of tissue damaging free radicals upon reperfusion with oxygen rich blood. [22] The ensuing inflammatory cascade results in recruitment of adhesive leukocytes and aggregation of SS erythrocytes and vascular blockage. [23] Therapies that disrupt the oxidative stress/inflammation feed back loop and have great potential as treatments for SCD. [24]

### BRIEF SUMMARY OF THE FIGURES

FIG. 1 shows the results of hypoxia/reoxygenation experiments in NY1DD mice, as observed by intravital microscopic analysis of cremastic venules. Values are mean±standard deviation, (n=6 animals; 15 measurements/animal). A single i.p. dose of pyridoxamine (400 mg/kg) leads to: (a) 24% decrease in rolling leukocytes; (b) 84% decrease in leukocyte adhesion; (c) 95% decrease in leukocyte emigration; (d) 42% increase in red blood cell flow velocity (Vrbc); and (e) 92% increase in wall shear rate. Minimal change in (f) venule diameter is observed. Values are mean±standard deviation (n=6 animals; 15 measurements/animal). All p values <0.001.

FIG. 2 shows the results of magnetic resonance imaging experiments that demonstrate sustained improvements in tissue oxygenation and normalization of cerebral blood flow in Berkeley mice treated with a single oral dose of pyridoxamine (400 mg/kg; gavage). (a) brain images from a blood-oxygen-level dependent (BOLD) magnetic resonance imaging (MRI) study show baseline levels of deoxygenated Hemoglobin in a (i) untreated C57 wild type mouse, and (ii) untreated Berkeley mouse. Increasing percent (%) signal enhancement (red) corresponds to an increase in the deoxygenated hemoglobin/oxygenated hemoglobin ratio, which correlates with a decrease in tissue oxygenation. (iii) treated Berkeley mice 72 h after treatment with a single oral dose of pyridoxamine demonstrates a significant decrease in the deoxygenated hemoglobin/oxygenated hemoglobin ratio and increase in tissue oxygenation, with maximum effect occurring at 72 h (b) Significant differences in cerebral blood flow between an (i) untreated C57 wild type mouse, and (ii) untreated Berkeley mouse. A single oral dose of pyridoxamine normalized the CBF to near wild type in Berkeley mice, with maximum effect occurring after 72 h (iii). Values are mean±standard deviation (n=6 animals; 15 measurements/animal). All p values <0.001.

FIG. 3 shows that a single i.p. dose of pyridoxamine (400 mg/kg) causes a 95% decrease in leukocyte emigration. Values are mean±standard deviation (n=6 animals; 15 measurements/animal). All p values <0.001

FIG. 4 shows that a single i.p. dose of pyridoxamine (400 mg/kg) causes a 42% increase in RBC velocity. Values are mean±standard deviation (n=6 animals; 15 measurements/animal). All p values <0.002.

FIG. 5 shows that a single i.p. dose of pyridoxamine (400 mg/kg) causes a 92% increase in shear rate. Values are mean±standard deviation (n=6 animals; 15 measurements/animal). All p values <0.001.

FIG. 6 shows that a single i.p. dose of pyridoxamine (400 mg/kg) caused minimal change in venule diameter. Values are mean±standard deviation (n=6 animals; 15 measurements/animal). All p values <0.001.

FIG. 7 shows the change in whole brain hyperoxia induced BOLD signal following administration of pyridoxamine (400 mg/kg; gavage). An improvement in hemoglobin oxygenation is seen, as evidenced by the trend toward reduced deoxyhemoglobin/oxyhemoglobin ratio, with maximal effect at 72 hours (p=0.024). The difference between BERK and WT was significant only prior to treatment (indicated p<0.05).

FIG. 8 shows a MRI BOLD signal (decimal percent change) during hyperoxia challenge. A decrease indicates reduction in tissue deoxyhemoglobin/oxyhemoglobin ratio. Oxyhemoglobin levels increase in cranial muscle at different time points following oral administration of pyridoxamine (400 mg/kg; gavage), with maximal effect seen at 72 h. Indicated p<0.001.

FIG. 9 shows the effect of a single dose of pyridoxamine (400 mg/kg; gavage) upon BERK/WT (untreated) whole brain blood flow ratio. Quantitative perfusion measurements were obtained using a segmented and interleaved continuous arterial spin labeled (FAIR-Quipps) sequence employing a separate coil for arterial labeling. Published methods were used to extract perfusion values. Appropriate modification of the published method for the underlying tissue T1, was used for both brain and muscle perfusion. p<0.01 for a difference between BERK and WT (n=3 in each group).

FIG. 10 shows the effect of a single dose of pyridoxamine (400 mg/kg; gavage) upon BERK vs. WT (untreated) cranial muscle blood flow. Quantitative perfusion measurements were obtained using a segmented and interleaved continuous arterial spin labeled (FAIR-Quipps) sequence employing a separate coil for arterial labeling. Published methods were used to extract perfusion values. Appropriate modification of the published method for the underlying tissue T1, was used for both brain and muscle perfusion. p<0.01 for a difference between BERK and WT (n=3 in each group).

FIG. 11 shows MRI images demonstrating sustained improvements in tissue oxygenation and normalization of cerebral blood flow (CBF) in BERK mice treated with a single oral dose of pyridoxamine (400 mg/kg; gavage). (a) brain images from a BOLD MRI study show baseline levels of deoxyHb in a (i) untreated C57 wild type mouse, and (ii) untreated BERK mouse. Increasing percent (%) signal enhancement (lightened areas) corresponds to an increase in the deoxyHb/oxyHb ratio, which correlates with a decrease in tissue oxygenation. (iii) treated BERK 72 h after treatment with a single oral dose of NM-96 demonstrates a significant decrease in the deoxyHb/oxyHb ratio and increase in tissue oxygenation (darkened areas), with maximum effect occurring at 72 h (b) Significant differences in CBF between an (i) unstreated C57 wild type mouse, and (ii) untreated BERK mouse. A single oral dose of NM-96 normalized the CBF to near wild type in BERK mice, with maximum effect occurring after 72 h.

### DETAILED DESCRIPTION OF THE INVENTION

The following terms shall be used throughout the specification to describe the present invention. Where a term is not specifically defined herein, that term shall be understood to be used in a manner consistent with its use by those of ordinary skill in the art.

Where a range of values is provided in the present application, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention. In instances where a substituent is posited for use in a present compound, it is understood that only those substituents which form stable bonds or stable compounds are to be embraces.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal, especially including a human, to whom treatment, including prophylactic treatment (prophylaxis), with the compounds or compositions according to the present invention is provided. For treatment of those infections, conditions or disease states which are specific for a specific animal such as a human patient, the term patient refers to that specific animal. In most instances, the patient or subject of the present invention is a human patient of either or both genders. In certain embodiments, the patient is resistant to therapy with hydroxyurea. In other embodiments, hydroxyurea may be coadministered with pyradoxamine to effect therapy in the patient. The term “prophylactic” or “prophylaxis” shall mean preventing or reducing the likelihood that a disease, condition or event will occur.

The term “effective” is used herein, unless otherwise indicated, to describe an amount of a compound or component which, when used within the context of its use, produces or effects an intended result, whether that result relates to the prophylaxis and/or therapy of a disease state, a secondary disease state or condition thereof or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers (including diasteromers) if applicable, individual optical isomers (enantiomers) or racemic mixtures, pharmaceutically acceptable salts, prodrug forms, including hydrates and solvates of these compounds. The term compound herein refers to stable compounds. Within its use in context, the term compound may refer to a single compound or a mixture of compounds as otherwise described herein.

The term “bioactive agent” or “additional bioactive agent” refers to any biologically active compound or drug which may be formulated for use in an embodiment of the present invention. Exemplary bioactive agents include the compounds according to the present invention which are used to treat sick cell anemia, Thallesemia or a disease state or condition which occurs secondary to sick cell anemia, thallesemia or and other related blood diseases as well as other compounds or agents which are otherwise described herein.

Bioactive agents for use in the present invention include anti-sickling agents, selectin and adhesion inhibitors, fetal hemoglobin regulators, agents which inhibit a platelet ADP receptor and other anti-platelet agents, agents which increase the affinity of sickle hemoglobin's binding to oxygen, antioxidants, nitric oxide generating agents, vascular tone agents and anti-inflammatory agents and agents that target leukocyte adhesion, among others. A fetal hormone regulator includes hemoglobin-inducing agents, in particular fetal hemoglobin-inducing agents.

The following presents specific representative bioactive agents which can be coadministered with pyridoxamine or a pharmaceutically acceptable salt thereof.

anti-sickling agents: 5-hydroxymethylfurfural, 4-Hydroxy-3-methoxybenzaldehyde.

selectin and adhesion inhibitors: GMI-1070, GMI-1271, intravenous immunoglobulin, tinzaparin, propranolol, SelG1 (humanized anti p-selectin antibody), heparin.

fetal hemoglobin regulators include hemoglobin inducing agents: hydroxyurea, decitabine, sodium dimethylbutyrate, pomalidomide, (2E)-N-hydroxy-3-[4-({[2-(2-methyl-1H-indol-3-yl)ethyl]amino}methyl)phenyl]acrylamide, sodium phenyl butyrate, sodium phenyl acetate, sodium phenyl propionate, or an alternative pharmaceutical salt thereof (as disclosed in U.S. Pat. No. 5,712,307, which is incorporated by reference herein), cyclic peptides such as FK228 (depsipeptide) and analogs thereof, as disclosed in Anemia 2012; 2012: 428137 (published online May 14, 2012), benzamides (such as MS-275); non cyclic and cyclic hydroxamates, for example SAHA (suberoylanilide hydroxamic acid) and TSA (Trichostatin A) among others.

agents which inhibit a platelet ADP receptor: (R,S)-5-[2-cyclopropyl-1-(2-fluorophenyl)-2-oxoethyl] 4,5,6,7-tetrahydrothieno[3,2-c]pyridin-2-yl acetate, N-hydroxy-N′-phenyl-octanediamide.

agents which increase the affinity of sickle hemoglobin's binding to oxygen: 5-hydroxymethylfurfural, pyridoxal-5-phosphate, 4-hydroxy-3-methoxybenzaldehyde.

antioxidants including, but not limited to, para-aminobenzoic acid (PABA), pyridoxine, pyridoxine-5-phosphate, pyridoxal-5-phosphate, ascorbic acid, N-acetyl cysteine, α-linolenic acid, eicosapentaenoic acid, docosahexanoc acid, glutamine, acetyssl-L-carnitine.

Nitric oxide generating agents: L-Arginine.

Vascular tone agents: intravenous magnesium.

Anti-inflammatory agents: 2-{4-[(methylamino)carbonyl]-1H-pyrazol-1-yl}adenosine, N-[1-(1-benzothien-2-yl)ethyl]-N-hydroxyurea, Fructose-1,6-diphosphate, oxooxan-2-yl]ethyl}-3,7-dimethyl-1,2,3,7,8,8a-hexahydronaphthalen-1-yl (2S)-2-methylbutanoate, 2-acetoxybenzoic acid.

The terms “treat”, “treating”, and “treatment”, are used synonymously to refer to any action providing a benefit to a patient at risk for or afflicted with a disease state or condition as described herein, including improvement in the disease state or condition through lessening, inhibition, suppression or elimination of at least one symptom, delay in progression of the disease, prevention, delay in or inhibition of the likelihood of the onset of the disease or condition, etc.

Treatment, as used herein, encompasses both prophylactic and therapeutic treatment, principally of sickle cell diseases, Thalassemia and other related blood diseases, as well as secondary disease states and conditions such as vessel occlusion, chronic and systemic vasculopathies, as well as stroke, sickle retinopathy, open leg ulcers, multi-organ failure, aseptic bone necrosis, dactylitis, hepatopathy, splenic autoinfarction, pulmonary hypertension, cognitive deficits, renal failure, cholecystitis, decreased fertility, increased susceptibility to infection and decreased opsonization, as well as other disease states and conditions. Compounds according to the present invention can, for example, be administered prophylactically to a patient in advance of the occurrence of a disease state or condition to reduce the likelihood of that disease state or condition. Prophylactic administration is effective to reduce or decrease the likelihood of the subsequent occurrence of disease in the patient, or decrease the severity of the disease state or condition that subsequently occurs, especially including secondary disease states or conditions.

Alternatively, compounds according to the present invention can, for example, be administered therapeutically to a patient that is already afflicted by disease. In one embodiment of therapeutic administration, administration of the present compounds is effective to eliminate the disease and substantially eliminate the likelihood of further manifestations of disease. Administration of the compounds according to the present invention is effective to decrease the severity of the disease or lengthen the lifespan of the mammal so afflicted, or inhibit or even eliminate the causative agent of the disease.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

The term “inhibit” as used herein refers to the partial or complete elimination of a potential effect, while inhibitors are compounds/compositions that have the ability to inhibit.

The term “prevention” or “prophylactic” when used in context shall mean “reducing the likelihood” or preventing a disease, condition or disease state from occurring as a consequence of administration or concurrent administration of one or more compounds or compositions according to the present invention, alone or in combination with another agent. It is noted that prophylaxis will rarely be 100% effective; consequently the terms prevention and reducing the likelihood are used to denote the fact that within a given population of patients or subjects, administration with compounds according to the present invention will reduce the likelihood or inhibit a particular condition or disease state (in particular, the worsening of a disease state such as the growth or metastasis of cancer) or other accepted indicators of disease progression from occurring.

The terms “coadminister” and “coadministration” are used synonymously to describe the administration of pyrodoxamine or a pharmaceutically acceptable salt and at least one additional bioactive agent (as otherwise described herein), which are administered in amounts or at concentrations which would be considered to be effective amounts at or about the same time. While it is preferred that coadministered compositions/agents be administered at the same time, agents may be administered at different times such that effective concentrations of both (or more) compositions/agents appear in the patient at the same time for at least a brief period of time. Alternatively, in certain aspects of the present invention, it may be possible to have each coadministered composition/agent exhibit its inhibitory effect at different times in the patient, with the ultimate result being the inhibition and treatment of sickle cell disease, Thalassemia or a related blood disease or disorder or a secondary disease state or condition thereof. Of course, when more than disease state or condition is present, the present compounds may be combined with other agents to treat that other disease state or condition as required.

The term “sickle cell disease” (SCD) as used herein refers to a hereditary blood disorder in which red blood cells assume an abnormal, rigid, sickle shape. Sickling of erythrocytes decreases the cells' flexibility and results in a risk of various life-threatening complications. Sickle cell anemia is a form of sickle cell disease.

Sickle-cell disease may lead to various acute and chronic complications, several of which have a high mortality rate. These include sickle cell crisis, vaso-occlusive crisis, splenic sequestration crisis, acute chest syndrome (ACS), aplastic crisis, haemolytic crisis, dactylitis, increased risk of severe bacterial infections, especially Streptococcus pneumonia and Haemophilus influenza, due to loss of functioning spleen tissue, stroke, cerebral infarction in children, cerebral haemorrhage in adults, silent stroke, causing no outward symptoms but associated with damage to the brain, cholelithiasis (gallstones) and cholecystitis, avascular necrosis (aseptic bone necrosis) of the hip and other major joints, decreased immune reactions due to hyposplenism, priapism, osteomyletis (bacterial bone infection, often from Salmonella), acute papillary necrosis (kidneys), leg ulcers, eye complications (background retinopathy, proliferative retinopathy, vitreous hemorrhages and retinal detachments), pregnancy complications (intrauterine growth retardation, spontaneous abortion, pre-eclampsis, chronic pain (even in the absence of acute vaso-occlusive pain), pulmonary hypertension, strain on the right ventricle and risk of heart failure, nephropathy, chronic renal failure due to nephropathy and cognitive deficits. SCD patients often suffer from kidney disease and/or kidney related conditions similar to those of diabetes patients. These disease states and/or conditions may be treated using the present invention as well.

The term “Thalassemia” is used herein to describe an inherited blood disorder in which the body makes an abnormal form of hemoglobin resulting in less hemoglobin than normal and far fewer circulating red blood cells, resulting in a mild or severe anemia. Thalassemia is often present as microcytic anemia. Thalassemia can cause significant complications, including iron overload, an enlarged spleen, susceptible to illness, bone deformities and cardiovascular illness, each of which may be improved and/or resolved by treatment of principal disease state. Thalassemia may confer a level of protection against malaria.

The term “sickle cell trait” is used herein to describe a condition in which a person has one abnormal allele of the hemoglobin beta gene (is heterozygous), but does not display the severe symptoms of sickle cell disease that occur in a person who has two copies of that allele (is homozygous).

Sickle cell disease (SCD) can be cured using hematopoietic stem cell transplants, and the severity of the disease can be reduced with repeated transfusions. However, these approaches are costly and associated with high levels of risk. The only FDA approved therapeutic for SCD is hydroxyurea (HU). HU induces fetal Hb (HbF) production, and has a strong clinical track record in children and adults. Proper use of HU requires strict compliance; self-administration by the patient; and careful monitoring by the physician to ensure proper dosing schedules. [2] Unfortunately, approximately a third of adult patients do not respond to HU treatment. [4] Moreover, myeolsuppression, reproductive toxicity and possible carcinogenic affects are all serious side effects associated with long-term use. [5] Importantly, there is currently no commercial therapeutic to avert imminent episodes of crisis or to attenuate acute events of crisis once they have begun. Patients are typically prescribed medications that alleviate the pain associated with episodes of crisis.

While SCD is typically characterized as a molecular disease; its pathophysiological consequences mimic those of ischemic reperfusion injury. [6] The chronic hemolytic anemia disrupts the oxidative stress equilibrium of patients, and can trigger inflammatory cascades that result in adhesion of leukocytes to vascular walls, recurrent and intermittent episodes of vaso-occlusion (VOC) and painful episodes of crisis. [4, 7] The pain associated with crisis can be debilitating, and lead to hospitalization. [3] Due to the importance of oxidative stress events in propagating VOC, many forms of antioxidant therapy are being actively pursued as novel approaches to treat the disease. Examples include oral supplementation of arginine [8], N-acetyl cysteine [9] and treatment with omega-3 fatty acids. [10]

Chronic hemolytic anemia and vaso-occlusive ischemia-reperfusion like injury are the hallmark pathologies of sickle cell disease. [5] Oxidative stress and NO homeostasis are thought to play a major role in the severity of the disease, and agents that modulate these pathways are highly desirable. The multifactorial process, which leads to VOC involves oxidative stress, damages to red blood cells (RBC), inflammation, vascular leukocyte adhesion, coagulation and abnormal rheology, and vascular tone modulation. [6] Ameliorating oxidative stress by directly targeting free radicals, reactive carbonyls and other oxidizing species with non-toxic therapeutic agents has tremendous potential as a treatment for SCD. Surprisingly, Nanometics (in collaboration with the Albert Einstein College of Medicine) has discovered that pyridoxamine is useful to ameliorate the effects of SCD in transgenic mouse models of disease. Treatment with pyridoxamine has sustained effects (for up to 9 days) that suggest a regenerative aspect to the therapeutic approach. Pyridoxamine can act by several potential anti-oxidative mechanisms; nucleophilic scavenging of reactive carbonyl species; trapping of free radicals; and chelation of radical generating metal ions. [18, 19]

While not wishing to be bound by theory, the operating hypothesis is that a single dose of pyridoxamine significantly alleviates oxidative stress events in the microcirculation, allowing the animal to correct oxidative imbalances and regenerate normal function. Thus, the effects of pyridoxamine administration are realized for several days after pyridoxamine has been cleared from the system. In NY1 DD mouse models of acute sickle crisis that are exposed to 16 hours of hypoxic stress, pyridoxamine preserved endothelial function and ameliorated the hypoxia-induced endothelial activation that precedes venular occlusion and episodes of sickle crisis. In these studies, significant improvements in leukocyte behavior and hemodynamic properties were observed. The preliminary results are encouraging, and support the hypothesis that pyridoxamine will be useful as a prophylactic therapy to restore normal microvascular function. It is further anticipated that normalization of oxidative homeostasis will improve the half-life of circulating erythrocytes by decreasing the likelihood of oxidative damage and hemolysis.

There are several emerging therapies that are under clinical evaluation and these include: fetal hemoglobin-inducing agents, agents that target leukocyte adhesion, anti-inflammatory agents, anti-oxidant therapies, anti-platelet therapies and anti-sickling approaches. [17] Pyridoxamine is unique in that it is a well-tolerated vitamer of the B6 family, and has been studied extensively in humans. Moreover, preliminary results suggest that pyridoxamine is multimodal; disrupting molecular mechanisms of the vaso-occlusion (VOC) cascade, and simultaneously facilitating oxygen delivery to the brain. Many of the emerging therapies target the VOC cascade, and are predominantly anti-inflammatory approaches. It is anticipated that pyridoxamine will be useful as a single agent, or as part of a combination therapy (co-administration approach) to treat sickle cell disease.

Such co-administration of current or emerging therapeutics with pyridoxamine may also permit administration of lower dosages of these other therapeutics, thus minimizing potential side effects. Thus, in a further aspect, the present invention provides pharmaceutical compositions comprising (a) pyridoxamine, or a pharmaceutically acceptable salt thereof; and (b) one or more compounds that can provide benefit in a human patient, or pharmaceutically acceptable salts thereof. In a preferred embodiment, such compounds are selected from the group consisting of fetal hemoglobin-inducing agents as otherwise described herein, agents that target leukocyte adhesion, anti-inflammatory agents, anti-oxidant therapies, anti-platelet therapies or anti-sickling therapeutics. In another preferred embodiment, pyridoxamine is co-administered with hydroxyurea.

The present invention provides pharmaceutical compositions of pyridoxamine, and methods for using such compositions in to treat human patients who have sickle cell disease, Thalassemia or a related blood disease.

In a first aspect, the present invention provides pharmaceutical compositions, comprising (a) 25 to 2000 milligrams of pyridoxamine, or a pharmaceutically acceptable salt thereof; and (b) a pharmaceutically acceptable carrier. Dosage unit forms of the pharmaceutical compositions of the present invention comprise between 25 mg and 2000 mg of pyridoxamine, or a pharmaceutically acceptable salt thereof. Such dosage unit forms can comprise, for example, 25, 50, 75, 100, 125, 150, 175, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or 2000 mg of pyridoxamine, or a pharmaceutically acceptable salt thereof, or any range of such dosage unit forms. In a preferred embodiment, the dosage unit forms of the pharmaceutical compositions comprise between 50 mg and 500 mg of pyridoxamine, or a pharmaceutically acceptable salt thereof. Such dosage unit forms can comprise, for example, 50, 75, 100, 125, 150, 175, 200, 250, 300, 350, 400, 450, or 500 mg of pyridoxamine, or a pharmaceutically acceptable salt thereof. The dosage unit form can be selected to accommodate the desired frequency of administration used to achieve a specified daily dosage of pyridoxamine, or a pharmaceutically acceptable salt thereof, to a patient in need thereof. Preferably the unit dosage form is prepared for once daily or twice daily administration to achieve a daily dosage of between 50 and 2000 mg, more preferably between 100 and 1000 milligrams.

Pharmaceutically acceptable salts in accordance with the present invention, are salts with physiologically acceptable bases and/or acids well known to those skilled in the art of pharmaceutical technique. Suitable salts with physiologically acceptable bases include, for example, alkali metal and alkaline earth metal salts, such as sodium, potassium, calcium and magnesium salts, and ammonium salts and salts with suitable organic bases, such as methylamine, dimethylamine, trimethylamine, piperidine, morpholine and triethanolamine. Suitable salts with physiologically acceptable acids include, for example, salts with inorganic acids such as hydrohalides (especially hydrochlorides or hydrobromides), sulphates and phosphates, and salts with organic acids. The pharmaceutical compositions of this aspect of the invention include admixtures of the pyridoxamine, or pharmaceutically acceptable salt thereof, and the one or more other compounds, as well as separate unit dosages of each that are manufactured for combinatorial use. Such separate unit dosages may be administered concurrently or sequentially as determined by the clinician.

In all aspects of the pharmaceutical compositions of the present invention, the compounds are combined with one or more pharmaceutically acceptable carriers appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art. In a preferred embodiment of each of the above aspects of the invention, the pharmaceutical compositions of the invention are prepared for oral administration. As such, the pharmaceutical composition can be in the form of, for example, a tablet, a hard or soft capsule, a lozenge, a cachet, a dispensable powder, granules, a suspension, an elixir, a liquid, or any other form reasonably adapted for oral administration. The pharmaceutical compositions can further comprise, for example, buffering agents. Tablets, pills and the like additionally can be prepared with enteric coatings. Unit dosage tablets or capsules are preferred. Pharmaceutical compositions suitable for buccal administration include, for example, lozenges comprising pyridoxamine, or a pharmaceutically acceptable salt thereof and a flavored base, such as sucrose, acacia tragacanth, gelatin, and/or glycerin. Liquid dosage forms for oral administration can comprise pharmaceutically acceptable emulsions, solutions, suspensions, syrups, and elixirs containing inert diluents commonly used in the art, such as water. Such compositions can also comprise, for example, wetting agents, emulsifying and suspending agents, and sweetening, flavoring, and perfuming agents.

Pharmaceutical compositions according to the present invention comprise an effective amount of pyrodoxamine or a pharmaceutically acceptable salt, optionally in combination with an additional bioactive agent as otherwise described herein formulated to effect an intended result (e.g. therapeutic and/or prophylactic result) formulated in combination with a pharmaceutically acceptable carrier, additive or excipient. Pharmaceutical compositions according to the present invention may also comprise an addition bioactive agent or drug as otherwise described herein.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g. orally, transdermally, topically, perineurally or parenterally, that is, by intravenous, subcutaneous, intraperitoneal, intrathecal or intramuscular injection, among others, including buccal, rectal and transdermal administration. Subjects contemplated for treatment according to the method of the invention include humans, companion animals, laboratory animals, and the like. The invention contemplates immediate and/or sustained/controlled release compositions, including compositions which comprise both immediate and sustained release formulations. This is particularly true when one or more different bioactive agents are used in the pharmaceutical compositions in combination with pyridoxamine or its pharmaceutically acceptable salt as otherwise described herein. Administration of these formulations once or twice a day is preferably contemplated.

Formulations containing the compounds according to the present invention may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, preferably in unit dosage forms suitable for simple administration of precise dosages.

Pharmaceutical compositions according to the present invention typically include a conventional pharmaceutical carrier or excipient and may additionally include other medicinal agents, carriers, adjuvants, additives and the like. Preferably, the composition is about 0.1% to about 85%, about 0.5% to about 75% by weight of pyridoxamine or its salt and optionally, an additional bioactive agent, with the remainder of the composition consisting essentially of suitable pharmaceutical excipients.

An injectable composition for parenteral administration (e.g. intravenous, intramuscular or intrathecal) will typically contain the compound in a suitable i.v. solution, such as sterile physiological salt solution. The composition may also be formulated as a suspension in an aqueous emulsion. Liquid compositions can be prepared by dissolving or dispersing the active agent(s) (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration as discussed above, such excipients include pharmaceutical grades of mannitol, lactose, starch, magnesium stearate, sodium saccharine, talcum, cellulose, glucose, gelatin, sucrose, magnesium carbonate, and the like, as described. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g. an excipient such as a saccharide or cellulose preparation, a binder such as starch paste or methyl cellulose, a filler, a disintegrator, and other additives typically used in the manufacture of medical preparations.

Methods for preparing dosage forms for use in the present invention are known or are apparent to those skilled in the art; for example, see Remington's Pharmaceutical Sciences (17th Ed., Mack Pub. Co., 1985). The composition to be administered will contain a quantity of the selected compound in a pharmaceutically effective amount for therapeutic use in a patient according to the present invention. These pharmaceutical compositions can be prepared by any suitable method that includes the step of bringing into association pyridoxamine, or a pharmaceutically acceptable salt thereof (and optionally the other compounds) and the pharmaceutically acceptable carrier. In general, the compositions are prepared by uniformly and intimately admixing the pyridoxamine, or a pharmaceutically acceptable salt thereof, with a liquid or finely divided solid carrier, or both, and then, if necessary, shaping the product. For example, preparation of tablets can comprise compressing or molding a powder or granule of the compound. Compressed tablets can be prepared by compressing, in a suitable machine, the compound in a free-flowing form, such as a powder or granules optionally mixed with a binding agent, lubricant, inert diluent and/or surface active/dispersing agent(s). Molded tablets can be made by molding, in a suitable machine, the powdered compound moistened with an inert liquid diluent.

In another aspect, the present invention provides methods for limiting the progression of end organ disease and/or complications in a human patient with sickle cell disease by administering to the patient an amount of pyridoxamine, or a pharmaceutically acceptable salt thereof, effective to limit the progression of end organ disease or complications in the sickle cell disease patient. In a preferred embodiment, the methods comprise administering the pharmaceutical compositions of the invention to the patient. Thus, a preferred embodiment of the method comprises administering between 50 and 2000 milligrams of pyridoxamine, or a pharmaceutically acceptable salt thereof, to the patient, more preferably between 100 and 1000 milligrams of pyridoxamine, or a pharmaceutically acceptable salt thereof, optionally in combination with an additional bioactive agent.

The following examples are provided to provide further description of the present invention. These examples are provided for purposes of description and are not to be construed to limit the present invention.

### Examples

Pyridoxamine can scavenge free radicals and the present inventors recognized the potential for this molecule to be useful as an intervention in sickle cell disease; where elevated oxidative stress contributes to morbidity in patients. The present inventors used magnetic resonance imaging (MRI) to study the impact of a single oral dose of pyridoxamine on cerebral oxygenation and perfusion in Berkeley (Berkeley) models of disease. Berkeley mice are excellent models of SCD that display severe pathology and similar features to human disease. A single oral dose of pyridoxamine (100-400 mg/kg) normalized perfusion in the brain of Berkeley mice to wild type values (C57BL mice), with maximum response occurring 72 hours after administration. Changes in perfusion were accompanied by a concomitant improvement in brain tissue oxygenation and decrease in the ratio of deoxyHb/oxyHb in the brain and surrounding muscle. The benefits of pyridoxamine treatment were further demonstrated with intravital microscopy, where a single intraperitoneal (i.p.) dose ameliorated the hypoxia/reoxygenation induced endothelial activation in Berkeley and NY1 DD mouse models of disease

The present invention is exemplified and further described in the following examples.

### Example 1

NY1DD mice (2×n=6) were exposed to hypoxia for 16 h (gas mixture=10% O₂, 0.5% CO₂ and balance N₂). One group of NY1 DD was then be given an intraperitoneal (i.p.) dose of pyridoxamine (400 mg/kg), dissolved in phosphate buffered saline (PBS), prior to 3 h reoxygenation in ambient conditions. The second group of NY1 DD mice was put through the same hypoxia/reoxygenation process and given vehicle (PBS) as a negative control. Healthy C57 BL/6J mice were used as healthy controls and were not exposed to hypoxia/reoxygenation stress. At the end of the reoxygenation period, the cremaster muscle was excised for intravital microscopy analysis. Intravital microscopy: Mice were anesthetized i.p. with 10% urethane and 2% α-chloralose in saline (6 ml/kg). The animals were tracheotomized and the left carotid artery cannulated to monitor systemic arterial pressure. The open cremaster muscle was prepared according to the method of Baez. [1] The surface of the open cremaster muscle was suffused with a bicarbonate Ringer's solution of the following millimolar composition: NaCl 135.0, KCl 5.0, NaHCO3 27.0, MgCl2 0.64 and glucose 11.6. pH was adjusted to 7.35-7.4 by continuous bubbling with 94.6% N2 and 5.6% CO₂. The osmolarity of the solution, as measured by a Microosmette (Precision Systems, Inc., Sudsbury, MA) was 320 mOsm which is similar to that reported for the mouse plasma. [2] The cremaster muscle preparation was allowed to stabilize for 30 min before the initiation of the experiment. The temperature of the suffusion solution (flow rate, 5-6 ml/min) bathing the cremaster was maintained at 34.5-35° C. and monitored by a telethermometer (YSI Inc., Yellowsprings, Ohio) during the entire experiment. Oxygen tension of the suffusion solution bathing the tissue was determined using a microoxygen electrode (model MI-730; Microelectrodes Inc., Bedford, N.H.). Microscopic observations were carried out using microscope (model BH-2; Olympus Corp., Lake Success, N.Y.) equipped with a television camera (5000 Series; Cohu, Inc., San Diego, Calif.) and a Sony U-matic video recorder (model V05800; Sony Corp., Teaneck, N.J.). Identification of venules and arterioles for observation was determined according to the published procedure. [3] Vessel luminal diameter was measured on-line by image shearing using an image shearing device (model 907; Instruments for Physiology and Medicine, San Diego, Calif.). Red cell velocity (Vrbc) was measured along the vessel centerline using the dual-slit photodiode technique of Wayland and Johnson (ref). The dual-slit photo detector was placed over the projected image of the vessel and on-line analysis of the optical signals was performed using a cross-correlator as described by Tompkins et al. [4] (Model 102 BF; instruments for Physiology and Medicine). Vrbc measurements were made using a water immersion ×40 objective and ×6.3 eyepiece. The wall shear rates were calculated from the mean Vrbc (Vmean) and vessel diameter. The centerline Vrbc was converted to the mean red cell velocity across the vessel diameter using a conversion factor of 1.6 (Vrbc/Vmean=1.6) originally described by Baker and Wayland. Shear rates along the wall of microvessel of a given internal diameter (D) were calculated using the relationship: Wall shear rate=8 Vmean/D. Estimates of volumetric flow rates (Q) were made from Vmean and the vessel cross-sectional area (π D²/4). Rolling leukocytes were defined as those leukocytes that distinctly roll along the endothelial surface. The rolling leukocytes were easily distinguished because of their lower velocity compared with that of leukocytes and red cells in the flow. Rolling leukocyte velocity (μm/s) represented the time required for a rolling leukocyte to traverse a given length of venule. An average of rolling velocities of approximately ten leukocytes per venule was determined by frame-by-frame analysis of video replay. Rolling leukocyte flux (cells per min) was determined as the number of leukocytes rolling through a given point in a vessel. A leukocyte was considered adherent if it remained stationary for longer than 30 seconds. Adherent leukocytes were counted along the length of a given venule and expressed as average number of cells per 100-μm length of the vessel. Emigrated leukocytes were determined as the number of interstitial leukocytes in the field of view adjacent (within 30 μm) to venules. Statistical Analysis: A total of 15 venules per animal were analyzed for various microcirculatory flow parameters. Statistical analysis of the data was performed using one-way ANOVA, followed by Newman-Keuls multiple comparisons. Comparisons between groups (control versus transgenic) were made using the Student's t test. Where tests for normality failed, or Bartlett's test for homogeneity of variance showed significant difference in the SD, nonparametric tests such as Kruskal-Wallis test for ANOVA or the Wilcoxon two-sample test were used. P<0.05 was considered significant. The statistical analysis was performed using GraphPad Prism 6.

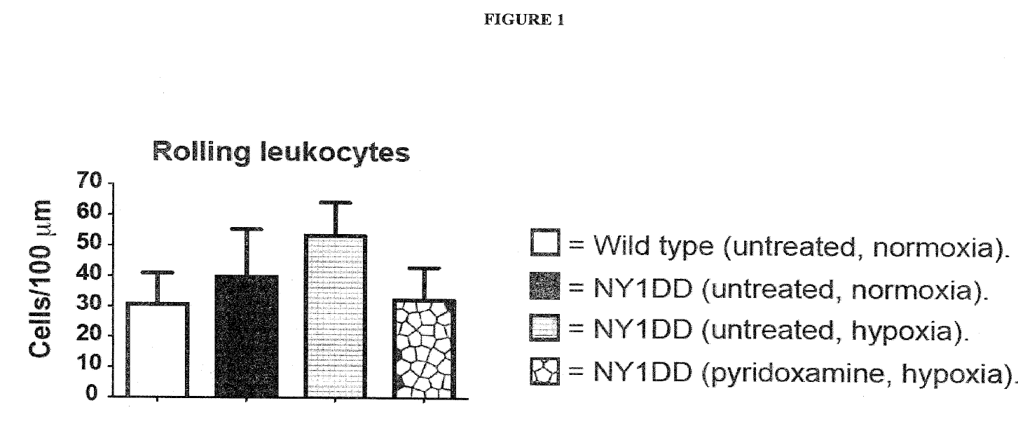

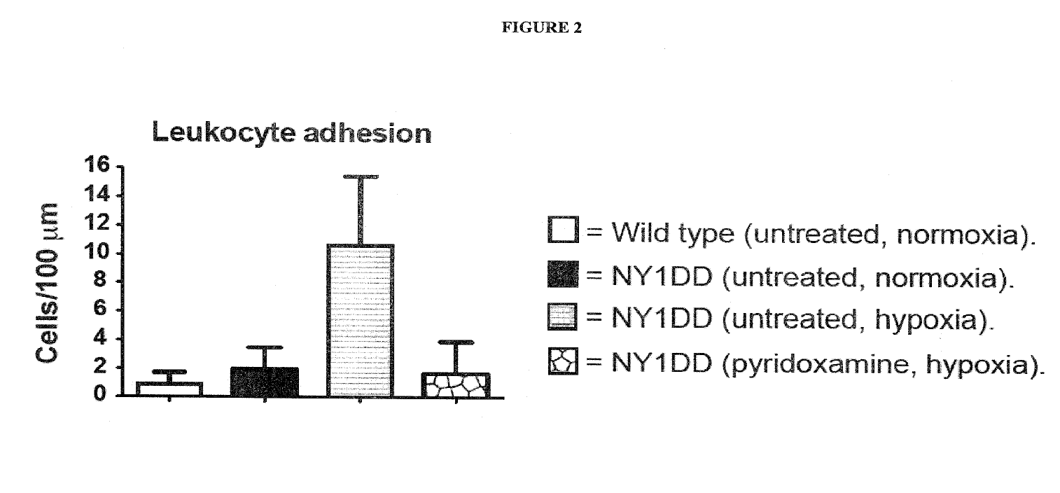

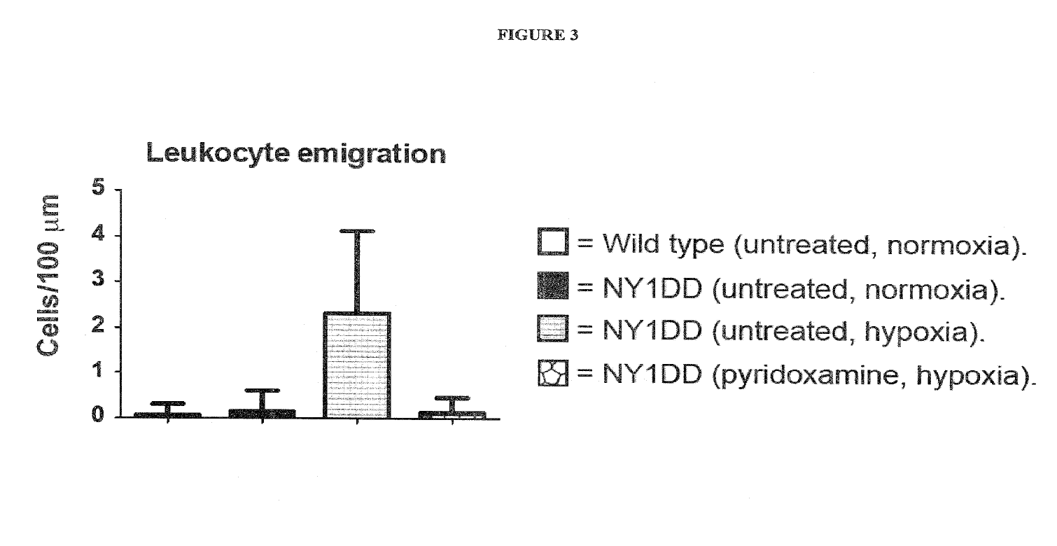

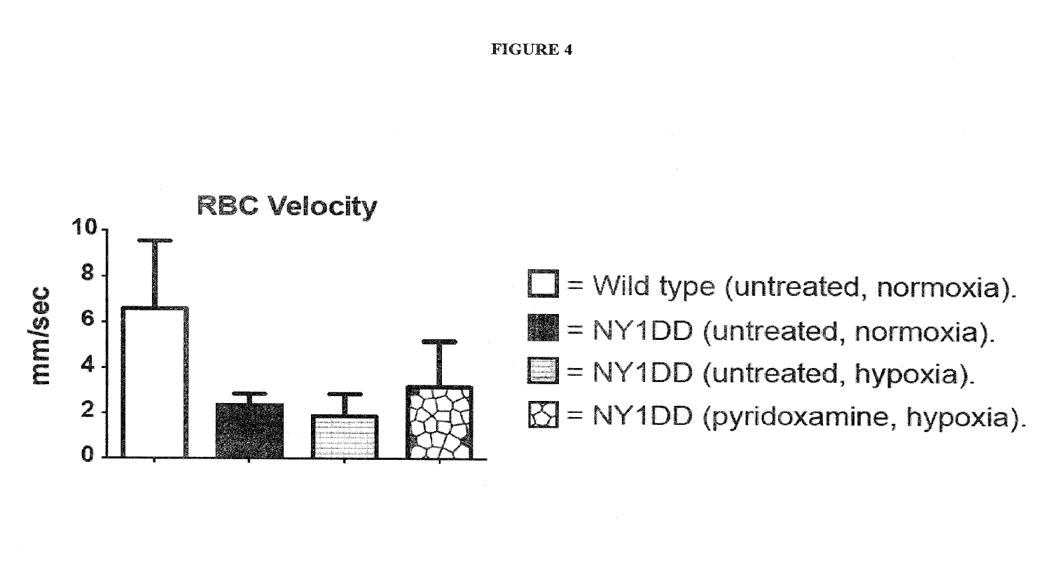

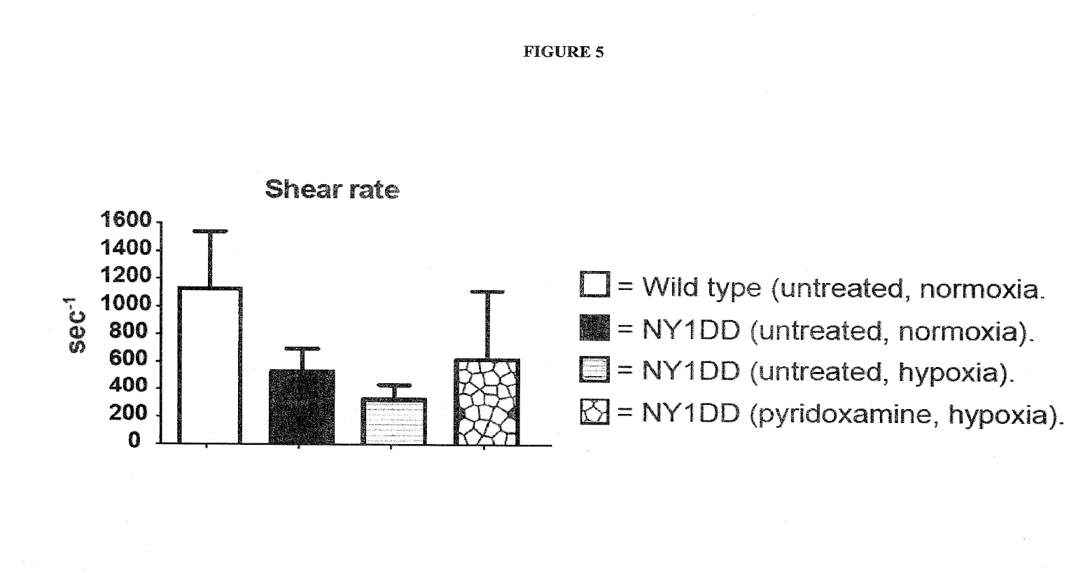

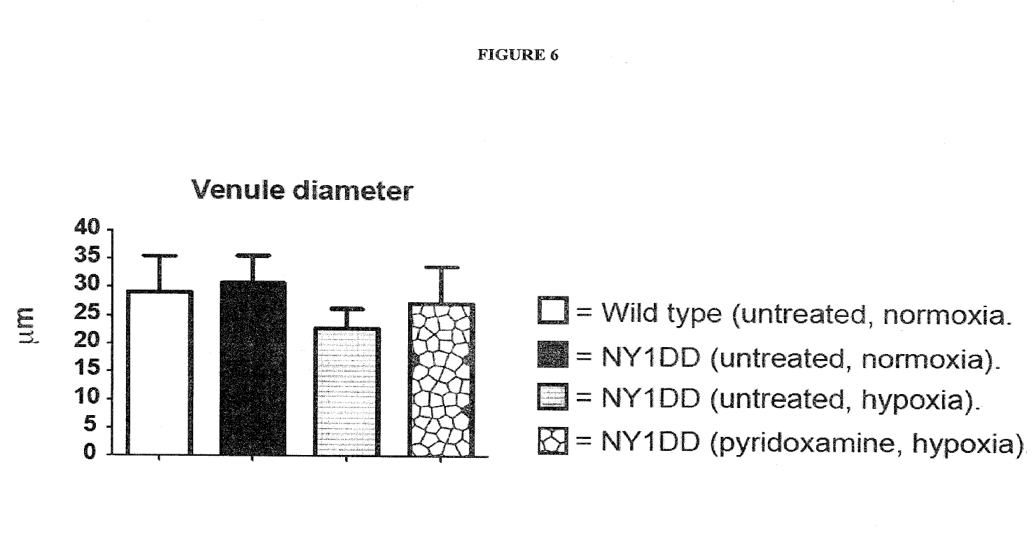

The results from the experiments in Example one are presented in attached FIGS. 1-6.

### Example 2

The experiments of Example 1 were repeated, except Berkeley low gamma mice were used.

### Example 3

The experiments of Example 1 were repeated, except Berkeley medium gamma mice were used to investigate the impact of increasing fetal hemoglobin concentrations and emulate a multimodal therapeutic strategy that would involve combinatorial administration of pyridoxamine and a fetal hemoglobin inducing agent.

### Example 4

The experiments of Example 1 were repeated, except Berkeley medium gamma mice were used to investigate the impact of increasing fetal hemoglobin concentrations and emulate a multimodal therapeutic strategy that would involve combinatorial administration of pyridoxamine and a fetal hemoglobin inducing agent.

### Example 5

A single oral dose of pyridoxamine normalizes cerebral function in Berkeley low gamma mouse models of sickle cell disease.

Major complications of sickle cell disease in humans, including asymptomatic cerebral infarction and stroke, are related to chronic abnormalities in cerebral blood flow. As in humans, Berkeley mice display abnormal cerebral blood flow, tissue oxygenation and higher ratios of deoxygenated hemoglobin/oxygenated hemoglobin. Here we demonstrate that a single oral dose of pyridoxamine can alleviate these abnormalities observed in Berkeley mice for several days after treatment. It is anticipated that chronic dosing with pyridoxamine will culminate in a normalization of brain physiology to near wild type (C57BL) levels. It is further anticipated that chronic dosing with pyridoxamine will also normalize the metabolism, as demonstrated by reduced lactic acid levels and improved N-acetyl aspartate (NAA)/choline ratios. Diffusion tensor imaging (DTI) can also be measured, and used to enable assessment for stroke/ischemia, and white matter tissue integrity by fractional anisotropy (FA). To identify focal pathology, quantitative MRI relaxometry and angiography will be performed.

Experimental Design & Methods: Dose Efficacy Experiment: A single oral dose (gavage) of pyridoxamine (400 mg/kg) was administered to BERK mice (n=6) or C57bl WT mice (n=4) and CBF and deoxyHb levels measured (see below for Measurement Parameters) before treatment and 2 h, 24 h, 72 h, 5 days and 9 days after treatment. Measurement Parameters: Each animal was assessed for several parameters by magnetic resonance imaging (MRI) methods on a 9.4 T Agilent MRI system. The primary MRI methods included brain and muscle perfusion, water diffusivity and oxygenation. Data acquisition was by multi-shot (8) Echo Planer Imaging (msEPI) (segmented and interleaved). Perfusion data were acquired using amultislice FAIR-Quipps approach, and blood oxygenation was determined from T2w msEPI (BOLD). Inflammation was inferred from the mean diffusivity of water calculated from a Diffusion Tensor Imaging (DTI) measurement which estimates the apparent diffusion coefficient of water (ADC). Transverse relaxation (T2) will be assessed for tissue free-water content using 30 echos (TE: 1.3 ms-240 ms) using multiecho-spin echo approaches. Longitudinal relaxation (T1) is assessed using EPI-inversion recovery (T1: 0.1 s-7 s, TR 8 s). Tissue Oxygenation: Bold Oxygen Level Dependent (BOLD) measurements were made during a hyperoxia challenge. MRI begins while animals are exposed to 20% O₂/80% N₂ (6 min) and imaging continues while the inspired gas is switched to 100% O₂, maintained for 6 minutes, then returned to normal room air levels. BOLD image signal intensity (echo-planer T2* weighted imaging) is inversely sensitive to deoxyHb levels, such that an increase in deoxyHb/oxyHb ratio causes a decrease in BOLD signal, while a decrease in this ratio causes an increase in BOLD signal intensity. Perfusion measurements: Quantitative perfusion measurements (FAIR-Quipps) employ a volume coil for arterial labeling, and surface coil for signal measurement, and a TI of 1200 ms. Water diffusion coefficient: DTI data were collected using a 7 direction assessment, from which both the mean diffusivity (DTI-MD) and fractional anisotropy (DTI-FA) were obtained. DTI-FA provides a measure of white matter integrity and DTI-MD, along with tissue T₂ provides a measure of tissue inflammation.

Data Analysis & Interpretation: All images are co-registered to a template using FSL tools (FMRIB Software library, Oxford, UK). The template was constructed of an averaged high-resolution averaged T2-weighted image from 3 co-registered BERK mice, or 3 co-registered C57bl mice. Regions of interest (ROI's) were drawn from standard atlases (Paxonis) on the template, and include white matter (CC and External Caudate), gray matter (Somatosensory, Motor, Frontal, Parietal) Cortex, hippocampus, basal ganglia, cingulate, and others) and cranial muscle from the anterior region of the head. Image data was converted from native format to NIFTI format, intensity flattened, phase corrected, orientation corrected, and registered to the template. Perfusion, BOLD activation ratios and metabolite ratios were calculated using MATLAB (Mathworks, Natick, Mass.). Parameter maps were calculated using a non-linear least squares in Matlab, or using the FMRIB FSL-based Diffusion Toolbox (DTIFIT). Registration was accomplished using the FSL-FLIRT routine. Published methods were used to extract perfusion values [32], with appropriate modification for the underlying tissue T₁, and a diffusion crushers to eliminate residual arterial signal from brain or muscle vasculature. ROI's were exported into excel spreadsheets for summary analysis.

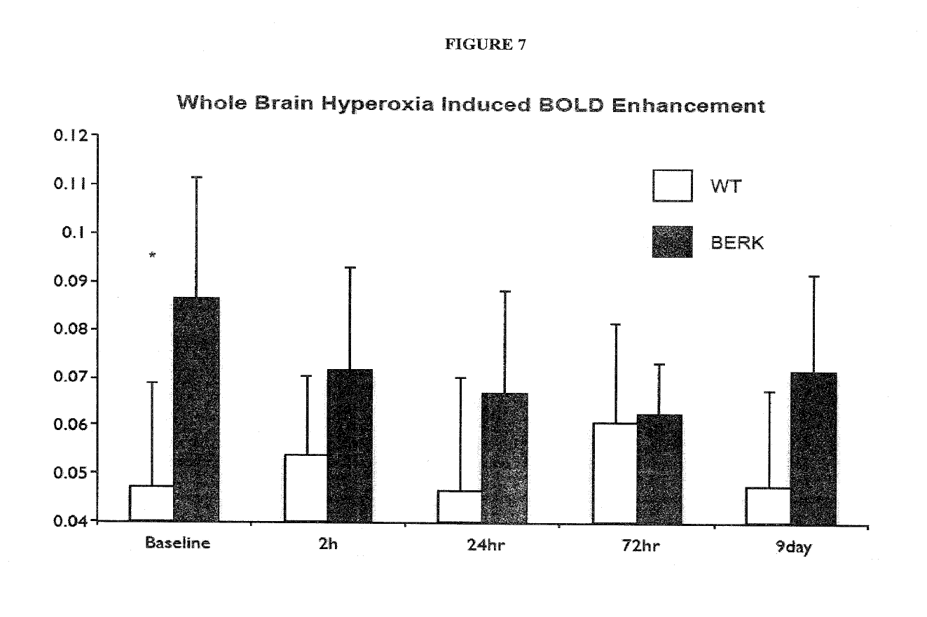

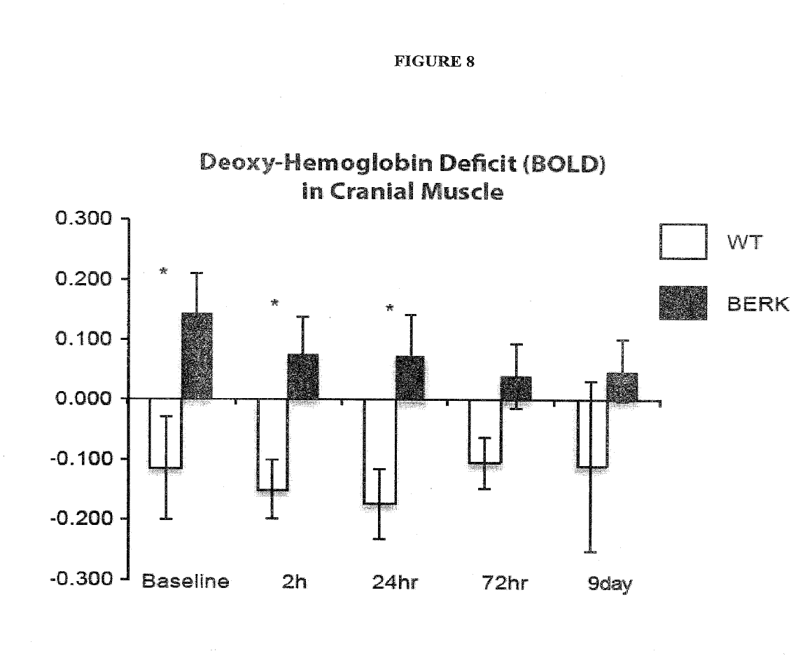

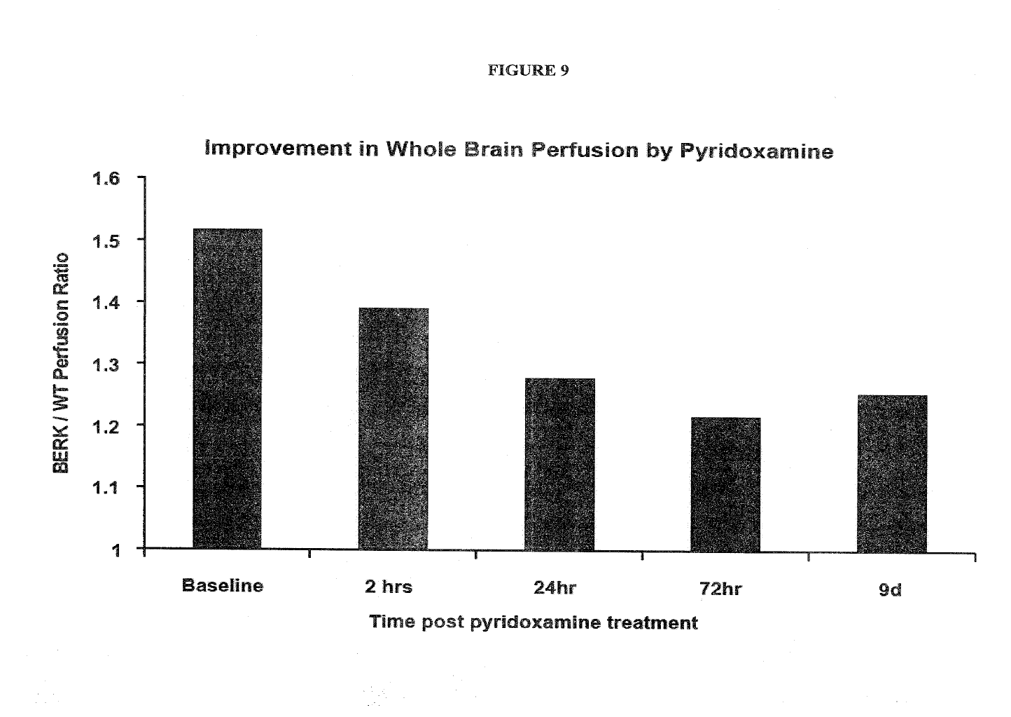

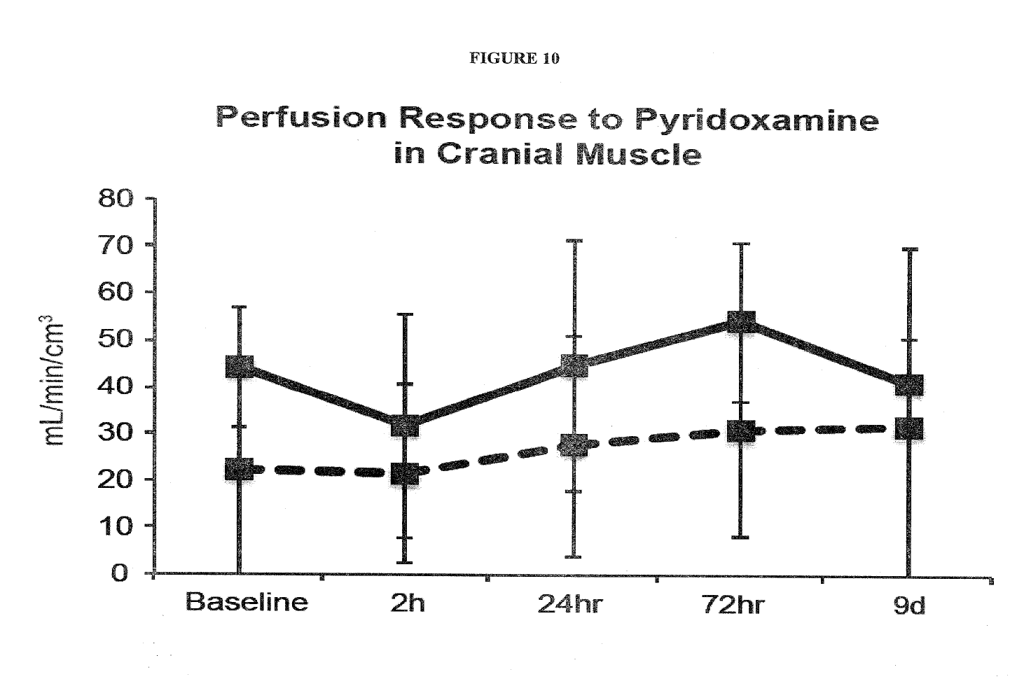

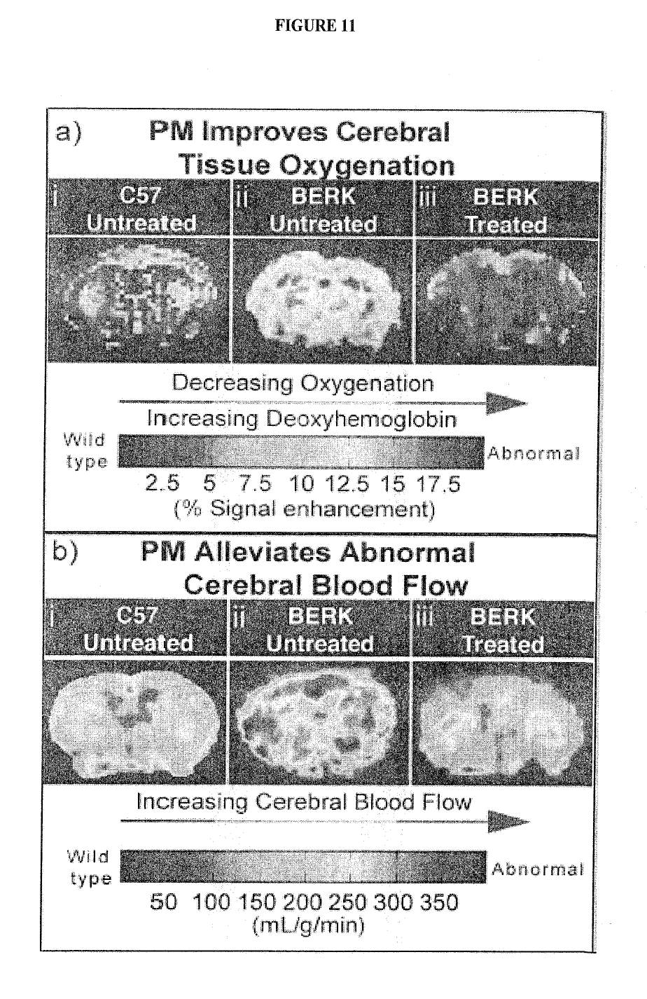

The results from Example 5 are presented in attached FIGS. 7-11.

### Example 6

A single oral dose of pyridoxamine normalizes cerebral function in Berkeley medium gamma mouse models of sickle cell disease.

The experiments of Example 5 were repeated, except Berkeley medium gamma mice were used to investigate the impact of increasing fetal hemoglobin concentrations and emulate a multimodal therapeutic strategy that would involve combinatorial administration of pyridoxamine and a fetal hemoglobin inducing agent.

### Example 7

A single oral dose of pyridoxamine normalizes cerebral function in Berkeley high gamma mouse models of sickle cell disease.

The experiments of Example 5 were repeated, except Berkeley high gamma mice were used to investigate the impact of increasing fetal hemoglobin concentrations and emulate a multimodal therapeutic strategy that would involve combinatorial administration of pyridoxamine and a fetal hemoglobin inducing agent.

1. A method for treating sickle cell disease, thalassemia or sickle cell trait or a secondary disease state or condition thereof in a human patient comprising orally administering to a human patient in need comprising an effective amount of a pyridoxamine, or a pharmaceutically acceptable salt thereof, optionally in combination with an additional bioactive agent.

2. The method of claim 1, wherein the patient is resistant to hydroxyurea therapy.

3. The method according to claim 1 wherein said patient is treated for sickle cell disease.

4. The method according to claim 1 wherein said patient is treated for thalassemia.

5. The method according to claim 1 wherein said patient is treated for sickle cell trait.

6. The method according to claim 1 wherein said secondary disease state or condition is venular occlusion, sickle crisis, chronic and/or systemic vasculopathies, stroke, sickle retinopathy, open leg ulcers, multi-organ failure, aseptic bone necrosis, dactylitis, hepatopathy, splenic autoinfarction, pulmonary hypertension, cognitive deficits, renal failure, cholecystitis, decreased fertility, increased susceptibility to infection and decreased opsonization.

7. The method according to claim 1 wherein said secondary disease state or condition is sickle cell crisis, vaso-occlusive crisis, splenic sequestration crisis, acute chest syndrome (ACS), aplastic crisis, haemolytic crisis, dactylitis, increased risk of severe bacterial infections, hyposplenism, stroke, cerebral infarction in children, cerebral haemorrhage in adults, silent stroke, cholelithiasis (gallstones) and cholecystitis, avascular necrosis of the hip and other major joints, decreased immune reactions, priapism, osteomyletis, acute papillary necrosis, leg ulcers, background retinopathy, proliferative retinopathy, vitreous hemorrhages and retinal detachments, intrauterine growth retardation, spontaneous abortion, pre-eclampsis, chronic pain, pulmonary hypertension, strain on the right ventricle and risk of heart failure, nephropathy, chronic renal failure due to nephropathy and cognitive deficits.

8. The method of claim 1 said pyridoxamine or a pharmaceutically acceptable salt thereof and optional additional bioactive agent is formulated in parenteral dosage form or sustained or controlled release oral dosage form.

9. The method according to claim 8 wherein said dosage form is administered to said patient once or twice a day.

10. The method according to claim 1 wherein said pyridoxamine, or a pharmaceutically acceptable salt thereof, is administered in oral dosage form in an amount ranging from about 50 mg to about 300 mg.

11. The method according to claim 10 wherein said pyridoxamine or a pharmaceutically acceptable salt thereof, is administered in a 50 mg. oral dosage form.

12. The method according to claim 10 wherein said pyridoxamine or a pharmaceutically acceptable salt thereof, is administered in a 250 mg. oral dosage form.

13. The method according to claim 10 wherein said pyridoxamine or a pharmaceutically acceptable salt thereof, is administered in a 300 mg. oral dosage form.

14. The method according to claim 11 wherein said oral dosage form is an immediate or sustained release dosage form administered to said patient once or twice a day.

15. The method according to claim 1 wherein said pyridoxamine or a pharmaceutically acceptable salt thereof is coadministered with an additional bioactive agent.

16. The method according to claim 1 wherein said additional bioactive agent is at least one agent selected from the group consisting of anti-sickling agents, selectin inhibitors, fetal hemoglobin regulators, inhibitors of a platelet ADP receptor and agents which increase the affinity of sickle hemoglobin's binding to oxygen.

17. The method according to claim 1 wherein said additional bioactive agent is at least one agent selected from the group consisting of anti-sickling agents, selectin and adhesion inhibitors, fetal hemoglobin regulators, agents which inhibit a platelet ADP receptor, agents which increase the affinity of sickle hemoglobin's binding to oxygen, anti-oxidants, nitric oxide generating agents, vascular tone agents, anti-inflammatory agents, agents that target leukocyte adhesion and anti-platelet agents.

18. The method according to claim 1 wherein said additional bioactive agent is 5-hydroxymethylfurfural, 4-Hydroxy-3-methoxybenzaldehyde, GMI-1070, GMI-1271, intravenous immunoglobulin, tinzaparin, propranolol, SelG1 (humanized anti p-selectin antibody), heparin, hydroxyurea, decitabine, sodium dimethylbutyrate, pomalidomide, (2E)-N-hydroxy-3-[4-({[2-(2-methyl-1H-indol-3-yl)ethyl]amino}methyl)phenyl]acrylamide, (R,S)-5-[2-cyclopropyl-1-(2-fluorophenyl)-2-oxoethyl] 4,5,6,7-tetrahydrothieno[3,2-c]pyridin-2-yl acetate, N-hydroxy-N′-phenyl-octanediamide, 5-hydroxymethylfurfural, pyridoxal-5-phosphate, 4-hydroxy-3-methoxybenzaldehyde, para-aminobenzoic acid (PABA), pyridoxine, pyridoxine-5-phosphate, pyridoxal-5-phosphate, ascorbic acid, N-acetyl cysteine, α-linolenic acid, eicosapentaenoic acid, docosahexanoc acid, glutamine, acetl-L-carnitine, L-Arginine, intravenous magnesium, 2-{4-[(methylamino)carbonyl]-1H-pyrazol-1-yl}adenosine, N-[1-(1-benzothien-2-yl)ethyl]-N-hydroxyurea, Fructose-1,6-diphosphate, oxooxan-2-yl]ethyl}-3,7-dimethyl-1,2,3,7,8,8a-hexahydronaphthalen-1-yl (2S)-2-methylbutanoate, 2-acetoxybenzoic acid or a mixture thereof.

19. The method according to claim 1 wherein said additional bioactive agent is hydroxyurea, sodium phenyl butyrate, sodium phenyl acetate, sodium phenyl propionate, or an alternative pharmaceutical salt thereof (as disclosed in U.S. Pat. No. 5,712,307, which is incorporated by reference herein), FK228 (depsipeptide) or an analog thereof, a benzamide, a non-cyclic or cyclic hydroxamate, and TSA (Trichostatin A).

20. A method of treating kidney disease and/or a kidney related condition or disorder secondary to sickle cell disease, thalassemia, sickle cell trait or diabetes (I or II) in a patient in need comprising administering to said patient an effective amount of pyridoxamine, or a pharmaceutically acceptable salt thereof, optionally in combination with an additional bioactive agent.

21. The method according to claim 20 wherein said kidney disease and/or kidney related condition or disorder is nephropathy or renal failure.

22. The method according to claim 21 wherein said renal failure is chronic renal failure and said disease state occurs secondary to sickle cell disease, thalassemia or sickle cell trait and diabetes.

23. The method of claim 20 wherein said pyridoxamine or a pharmaceutically acceptable salt thereof and optional additional bioactive agent is formulated in parenteral (preferably intravenous) dosage form or sustained or controlled release oral dosage form.

24. The method according to claim 23 wherein said oral dosage form is administered to said patient once or twice a day.

25. The method according to claim 24 wherein said oral dosage form comprises pyridoxamine, or a pharmaceutically acceptable salt thereof, and is administered in an amount ranging from about 50 mg to about 750 mg.

26. The method according to claim 25 wherein said oral dosage form comprises pyridoxamine, or a pharmaceutically acceptable salt thereof, and is administered in an amount of about 600 mg.

27. A pharmaceutical composition comprising an effective amount of pyridoxamine or a pharmaceutically acceptable salt thereof, in combination with a pharmaceutically acceptable carrier, additive and/or excipient, optionally in combination with an additional bioactive agent.

28. The composition according to claim 27 wherein said additional bioactive agent is at least one agent selected from the group consisting of anti-sickling agents, selectin inhibitors, fetal hemoglobin regulators, agents which inhibit a platelet ADP receptor, agents which increase the affinity of sickle hemoglobin's binding to oxygen, anti-oxidant agents, anti-inflammatory agents, agents that target leukocyte adhesion and anti-platelet agents.

29. The composition according to claim 27 wherein said additional bioactive agent is 5-hydroxymethylfurfural, 4-Hydroxy-3-methoxybenzaldehyde, GMI-1070, GMI-1271, intravenous immunoglobulin, tinzaparin, propranolol, SelG1 (humanized anti p-selectin antibody), heparin, hydroxyurea, decitabine, sodium dimethylbutyrate, pomalidomide, (2E)-N-hydroxy-3-[4-({[2-(2-methyl-1H-indol-3-yl)ethyl]amino}methyl)phenyl]acrylamide, (R, 5)-5-[2-cyclopropyl-1-(2-fluorophenyl)-2-oxoethyl] 4,5,6,7-tetrahydrothieno[3,2-c]pyridin-2-yl acetate, N-hydroxy-N′-phenyl-octanediamide, 5-hydroxymethylfurfural, pyridoxal-5-phosphate, 4-hydroxy-3-methoxybenzaldehyde, para-aminobenzoic acid (PABA), pyridoxine, pyridoxine-5-phosphate, pyridoxal-5-phosphate, ascorbic acid, N-acetyl cysteine, α-linolenic acid, eicosapentaenoic acid, docosahexanoc acid, glutamine, acetl-L-carnitine, L-Arginine, intravenous magnesium, 2-{4-[(methylamino)carbonyl]-1H-pyrazol-1-yl}adenosine, N-[1-(1-benzothien-2-yl)ethyl]-N-hydroxyurea, Fructose-1,6-diphosphate, oxooxan-2-yl]ethyl}-3,7-dimethyl-1,2,3,7,8,8a-hexahydronaphthalen-1-yl (2S)-2-methylbutanoate, 2-acetoxybenzoic acid or a mixture thereof.

30.-37. (canceled)
